(19) United States
(12) Patent Application Publication
(10) Pub. No.: US 2023/0121362 A1
(43) Pub. Date: Apr. 20, 2023

(54) COSMETIC COMPOSITION FOR SKIN CARE

(71) Applicant: L'OREAL, Paris (FR)

(72) Inventors: Yangdong CHEN, Shanghai (CN); Yirui ZHU, Shanghai (CN)

(73) Assignee: L'OREAL, Paris (FR)

(21) Appl. No.: 17/907,481

(22) PCT Filed: Mar. 31, 2020
(86) PCT No.: PCT/CN2020/082248

§ 371 Date: Sep. 27, 2022

(51) Int. Cl. A61K 8/34; A61K 8/06; A61K 8/44; A61K 8/81; A61Q 19/00

(52) U.S. Cl. CPC A61K 8/342; A61K 8/062; A61K 8/442; A61K 8/8147; A61K 8/8188; A61Q 19/00; A61K 2800/10; A61K 2800/48

(57) ABSTRACT

A cosmetic composition in the form of an oil-in-water emulsion comprises (i) at least one solid fatty alcohol; (ii) at least one amino acid surfactant; and (iii) at least one gelling agent selected from hydrophilic acrylic homopolymers and amphiphilic acrylic polymers. A non-therapeutic method for caring for the skin comprises applying said cosmetic composition to the skin.

### TECHNICAL FIELD

The present invention relates to a cosmetic composition. In particular, the present invention relates to a cosmetic composition for caring for keratin materials, especially the skin.

### BACKGROUND ART

The development of formulations dedicated to caring for and/or making up the skin and/or lips, is permanent. Said formulations have to show satisfactory properties in terms of application, such as easy usage, and also in terms of sensory, such as hydration and/or moisturizing feeling after application.

The skin is the protective barrier for the human body. It protects the interior of the body from physical injury (such as trauma) and biological injury (such as bacteria, viruses or fungi). The skin of the human body comprises the dermis and the epidermis. The epidermis is the top most layer of the skin, and its superficial layer is called the stratum corneum.

Moisturization is provided to the skin by the water in the deep-lying layers and by perspiration. Cutaneous moisturization disorders, and especially skin dryness, are often observed with age and/or changes in climate. However, such conditions may also be manifested in young individuals.

A wide variety of cosmetic compositions have been used to care for the skin, in particular to provide moisturizing or hydration to the skin. These compositions typically contain lipophilic moisturizing agents that inhibit water loss via occlusion. These compositions can additionally comprise other skin beneficial agents such as vitamins and humectants. Addition of these ingredients can increase the moisturization of the skin. For example, efforts have been made to formulate compositions in the form of creams or emulsions. Thanks to the thick texture of the compositions, it is possible to provide to the skin moisturizing feeling via occlusion. The emulsions are usually in the form of water-in-oil emulsion.

However, these types of products are still not satisfying.

Consumers feel an unappealing greasy after application of the product. Moreover, due to the thick texture of the products, it is not easy to apply on the skin evenly.

There is thus still a need to formulate a stable composition for caring for the skin, which has good cosmetic properties, in particular moisturizing and/or hydration of the skin without greasy feeling.

Besides, the composition is expected to have a good texture and be easily spread on the skin during application.

It is therefore desirable to provide stable skin care compositions that present excellent texture and application as well as sensory characteristics.

### SUMMARY OF THE INVENTION

The Applicant has now discovered that it is possible to formulate such compositions having the desired properties as described above.

Specifically, the Applicant has discovered that it is possible to formulate stable compositions for caring for and/or making up of keratin materials, which present excellent texture and application as well as sensory characteristics.

Accordingly, in a first aspect, the present invention relates to a cosmetic composition in the form of an oil-in-water emulsion, comprising:
(i) at least one solid fatty alcohol;
(ii) at least one amino acid surfactant; and
(iii) at least one gelling agent selected from hydrophilic acrylic homopolymers and amphiphilic acrylic polymers.

The cosmetic composition of the present invention is in the form of an oil-in-water emulsion. Thus, said cosmetic composition comprises a continuous aqueous phase and a dispersed fatty phase.

In a second aspect, the present invention relates to a non-therapeutic method for caring for the skin, comprising applying the cosmetic composition according to the first aspect of the present invention to the skin.

The composition of the present invention is easily applied evenly on the skin, without long term massaging by fingers.

The cosmetic composition of the present invention presents excellent texture and application as well as sensory characteristics.

The composition according to the present invention as described above is stable over time at high temperature (45° C.), for example, after storage for 2 months, and further, for example, for 3 months.

Other advantages of the present invention will emerge more clearly on reading the description and the examples that follow.

### DETAILED DESCRIPTION OF THE INVENTION

According to the first aspect, the present invention relates to a cosmetic composition in the form of an oil-in-water emulsion, comprising:
(i) at least one solid fatty alcohol;
(ii) at least one amino acid surfactant; and
(iii) at least one gelling agent selected from hydrophilic acrylic homopolymers and amphiphilic acrylic polymers.

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “ between...and” and “ranging from ... to ...”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

All percentages in the present invention refer to weight percentage, unless otherwise specified.

### Solid Fatty Alcohols

According to the first aspect, the cosmetic composition of the present invention comprises at least one solid fatty alcohol.

By “solid fatty alcohol”, it is meant a fatty alcohol, linear or branched, saturated or unsaturated, that is solid at room temperature (25° C.) and at atmospheric pressure (780 mmHg or 1 atm.). Fatty alcohols are water-insoluble, i.e. they have solubility in water of less than 1% by mass and preferably less than 0.5% by weight.

Preferably, the solid fatty alcohols are of structure R—OH with R denoting a saturated or unsaturated, linear alkyl group, optionally substituted with one or more hydroxyl groups, comprising from 14 to 30 carbon atoms.

Preferably, the solid fatty alcohol is selected from those having from 14 to 30 carbon atoms.

Preferably, solid fatty alcohol having from 14 to 30 carbon atoms may be selected from myristyl alcohol (1-tetradecanol), cetyl alcohol (1-hexadecanol), palmitoleyl alcohol (cis-9-hexadecen-1-ol), stearyl alcohol (1-octadecanol), arachidyl alcohol (1-eicosanol), behenyl alcohol (1-docosanol), erucyl alcohol (cis-13-docosen-1-ol), lignoceryl alcohol (1-tetracosanol), ceryl alcohol (1-hexacosanol), myricyl alcohol and melissyl alcohol (1-triacontanol).

More preferably, according to the present invention, the solid fatty alcohol is selected from alcohols having from 14 to 22 carbon atoms, such as cetyl alcohol (1-hexadecanol), stearyl alcohol (1-octadecanol), arachidyl alcohol (1-eicosanol), behenyl alcohol (1-docosanol) and mixtures thereof.

More preferably, the solid fatty alcohol is selected from cetyl alcohol (1-hexadecanol), stearyl alcohol (1-octadecanol), arachidyl alcohol (1-eicosanol), behenyl alcohol (1-docosanol) and mixtures thereof.

Mentions of solid fatty alcohols may be cetyl alcohol sold under the name Lanette® 16 by BASF, or behenyl alcohol sold under the name Lanette® 22 by the company BASF, or a mixture thereof.

Advantageously, the solid fatty alcohol is present in an amount ranging from 0.1% to 10%, preferably from 0.2% to 5% by weight, more preferably from 0.5% to 3% by weight, relative to the total weight of the composition.

### Amino Acid Surfactant

According to the first aspect, the composition of the present invention comprises at least one amino acid surfactant.

In one embodiment, said amino acid surfactant is derived from a carboxylate salt of amino acid wherein the amine group situated on the a-carbon or β-carbon of an amino acid salt is acylated with a C₈ to C₂₂ fatty acid derivative.

The carboxylate salts of these amino acids can be formed by conventional means such as by neutralization of the respective amino acid with a base. The amine group situated on the a-carbon or β-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈ to C₂₂ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in U.S. Pat. Application Publication No. 2008/0200704, published on Aug. 21, 2008, which application is incorporated herein by reference.

In one embodiment, said amino acid surfactant is represented by the formula (A):

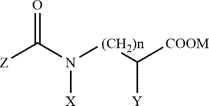

wherein:
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH_{z}OH, —CH(OH)CH₃, —(CH₂)₄NH₂, —(CH₂)₃NHC(NH)NH₂, —CH_{z}C(O)O—M⁺, —(CH₂)₂C(O)OH, —(CH₂)₂C(O)O—M+, and
M is a salt-forming cation, such as for example sodium, potassium, ammonium, or triethanolamine.

In one embodiment, in formula (A):
Z represents a linear or branched C₈ to C₂₂ alkyl or alkenyl group,
X is a hydrogen or methyl group,
n is 0,
Y is selected from hydrogen, —(CH₂)₂C(O)OH, —(CH₂)₂C(O)O—M⁺, and
M is a salt-forming cation, such as for example sodium, potassium, ammonium, or triethanolamine.

According to a preferred embodiment of the present invention, in the amino fatty acid of formula (A):
Z represents a linear or branched C₈ to C₂₂ alkyl or alkenyl group,
X is a hydrogen or methyl group,
n is 0,
Y is selected from hydrogen, —(CH₂)₂C(O)OH, —(CH₂)₂C(O)O″M⁺, and
M is a salt-forming cation, such as for example sodium, potassium, ammonium, or triethanolamine.

Examples of the amino acid surfactants are salt of alanine, arginine, aspartic acid, glutamic acid, glycine, isoleucine, leucine, lysine, phenylalanine, serine, tyrosine, valine, sarcosine, and any mixture thereof.

More specifically, mentions can be made of the amino acid surfactants such as dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, cocoyl methyl β-alaninate, lauroyl β-alaninate, lauroyl methyl β-alaninate, myristoyl β-alaninate, potassium lauroyl methyl β-alaninate, sodium cocoyl alaninate, sodium cocoyl methyl β-alaninate and sodium myristoyl methyl β-alaninate palmitoyl glycinate, sodium lauroyl glycinate, sodium cocoyl glycinate, sodium myristoyl glycinate, potassium lauroyl glycinate, potassium cocoyl glycinate, potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate ammonium lauroyl sarcosinate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

More preferably, the amino acid surfactant used in the composition is selected from salts of glutamic acid, in particular, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate.

Preferably, the amino fatty acid surfactant is present in the composition in an amount ranging from 0.1% to 5% by weight, preferably from 0.2% to 3% by weight, more preferably from 0.3% to 2% by weight, relative to the total weight of the composition.

### Hydrophilic Gelling Agents

According to the first aspect, the cosmetic composition of the present invention comprises at least one gelling agent selected from hydrophilic acrylic homopolymers and amphiphilic acrylic polymers.

### a-Hydrophilic Acrylic Homopolymers

According to the invention, the term “hydrophilic acrylic homopolymers” especially means non-hydrophobic and non-amphiphilic acrylic homopolymers.

The presence of this hydrophilic acrylic homopolymer makes it possible especially to obtain a composition that has good stability properties.

According to an embodiment, the hydrophilic acrylic homopolymer comprises at least one monomer bearing a sulfonic group.

The homopolymers used in accordance with the invention are homopolymers that may be obtained from an ethylenically unsaturated monomer bearing a sulfonic group, which may be in free form or partially or totally neutralized form.

Preferentially, the homopolymers are partially or totally neutralized with a mineral base (sodium hydroxide, potassium hydroxide or aqueous ammonia) or an organic base such as monoethanolamine, diethanolamine, triethanolamine, an aminomethylpropanediol, N-methylglucamine, basic amino acids, for instance arginine and lysine, and mixtures of these compounds. They are generally neutralized.

In the present invention, the term “neutralized” means homopolymers that are totally or virtually totally neutralized, i.e. at least 90% neutralized.

The homopolymers used in the composition of the invention generally have a number-average molecular weight ranging from 1000 to 20 000 000 g/mol, preferably ranging from 20 000 to 5 000 000 g/mol and even more preferentially from 100 000 to 1 500 000 g/mol.

These homopolymers according to the invention may be crosslinked or noncrosslinked. The monomers bearing a sulfonic group of the homopolymer used in the composition of the invention are especially chosen from vinylsulfonic acid, styrenesulfonic acid, (meth)acrylamido(C1-C22)alkylsulfonic acids, N-(C1-C22)alkyl(meth)acrylamido(C1-C22)alkylsulfonic acids such as undecylacrylamidomethanesulfonic acid, and also partially or totally neutralized forms thereof, and mixtures thereof.

According to one preferred embodiment of the invention, the monomers bearing a sulfonic group are chosen from (meth)acrylamido(C1-C22)alkylsulfonic acids, for instance acrylamidomethanesulfonic acid, acrylamidoethanesulfonic acid, acrylamidopropanesulfonic acid, 2-acrylamido-2-methylpropanesulfonic acid, 2-methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid and 2-acrylamido-2,6-dimethyl-3-heptanesulfonic acid, and also partially or totally neutralized forms thereof, and mixtures thereof.

More particularly, 2-acrylamido-2-methylpropanesulfonic acid (AMPS®), and also partially or totally neutralized forms thereof, is used.

When the homopolymers are crosslinked, the crosslinking agents may be chosen from the polyolefinically unsaturated compounds commonly used for crosslinking polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol% to 10 mol% and more particularly from 0.2 mol% to 2 mol% relative to the homopolymer.

The homopolymer of monomers bearing a sulfonic group may be crosslinked with one or more crosslinking agents.

These homopolymers are generally crosslinked and neutralized, and they may be obtained according to the preparation process comprising the following steps:
(a) the monomer such as 2-acrylamido-2-methylpropanesulfonic acid in free form is dispersed or dissolved in a solution of tert-butanol or of water and tert-butanol;
(b) the monomer solution or dispersion obtained in (a) is neutralized with one or more mineral or organic bases, preferably aqueous ammonia, in the amount making it possible to obtain a degree of neutralization of the sulfonic acid functions of the homopolymer ranging from 90% to 100%;
(c) the crosslinking monomer(s) are added to the solution or dispersion obtained in (b);
(d) a standard free-radical polymerization is performed in the presence of free-radical initiators at a temperature ranging from 10 to 150° C.; the homopolymer precipitates in the tert-butanol-based solution or dispersion.

The preferred AMPS homopolymers are generally characterized in that they comprise, randomly distributed:
a) from 90% to 99.9% by weight of units of general formula (l) below:

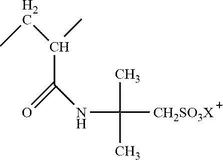

in which X⁺ denotes a proton, an alkali metal cation, an alkaline-earth metal cation or the ammonium ion, not more than 10 mol% of the cations X⁺ possibly being protons H⁺;
b) from 0.01% to 10% by weight of crosslinking units derived from at least one monomer containing at least two olefinic double bonds; the weight proportions being defined relative to the total weight of the homopolymer.

The homopolymers according to the invention that are more particularly preferred comprise from 98% to 99.5% by weight of units of formula (l) and from 0.2% to 2% by weight of crosslinking units.

A homopolymer of this type that may especially be mentioned is the crosslinked and neutralized 2-acrylamido-2-methylpropanesulfonic acid homopolymer sold by the company Clariant under the trade name Hostacerin® AMPS (CTFA name: ammonium polyacryldimethyltauramide).

### b- Amphiphilic Acrylic Polymers

Such polymers may be derived from the AMPS® products described previously.

These polymers comprise both a hydrophilic part and a hydrophobic part comprising at least one fatty chain. They are therefore amphiphilic polymers.

A fatty chain of such a polymer may comprise from 7 to 30 carbon atoms and in particular from 8 to 22 carbon atoms.

AMPS® copolymers in accordance with the invention may have a weight-average molecular weight ranging from 50 000 to 10 000 000, in particular from 100 000 to 8 000 000 and more particularly from 100 000 to 7 000 000.

AMPS® copolymers according to the invention may be crosslinked or noncrosslinked.

Among the crosslinking agents that may be suitable for use, mention may be made, in a nonlimiting manner, of methylenebisacrylamide, allyl methacrylate and trimethylolpropane triacrylate (TMPTA).

The degree of crosslinking may range from 0.01 mol% to 10 mol% and particularly from 0.2 mol% to 2 mol% relative to the polymer.

An amphiphilic AMPS® polymer that is suitable for use in the invention may be chosen, for example, from statistical amphiphilic AMPS polymers modified by reaction with a C6-C22 n-monoalkylamine or di-n-alkylamine such as those described in patent application WO 00/31154.

These polymers may also contain other ethylenically unsaturated hydrophilic monomers chosen, for example, from acrylic acid, methacrylic acid or alkyl-substituted derivatives thereof or esters thereof obtained with mono- or polyalkylene glycols, acrylamide, methacryl amide, vinylpyrrolidone, itaconic acid and maleic acid, or mixtures thereof.

A polymer of the invention may be chosen from amphiphilic polymers of AMPS® and of at least one ethylenically unsaturated monomer comprising at least one hydrophobic part containing from 7 to 30 carbon atoms, in particular from 8 to 22 carbon atoms and more particularly from 12 to 20 carbon atoms.

The hydrophobic part may be a saturated or unsaturated linear (for example n-octyl, n-decyl, n-hexadecyl, n-dodecyl or oleyl), branched (for example isostearic) or cyclic (for example cyclododecane or adamantane) alkyl radical.

An ethylenically unsaturated hydrophobic monomer that is suitable for use in the invention may be chosen from the acrylates or acrylamides of formula (ll) below:

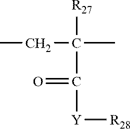

in which:
R₂₇ denotes a hydrogen atom or a linear or branched C₁-C₆ alkyl radical (preferably methyl),
Y denotes O or NH;
R₂₈ denotes a hydrophobic radical comprising a fatty chain containing from 7 to 30 carbon atoms, preferably from 8 to 22 and more particularly from 12 to 20 carbon atoms.

The hydrophobic radical R₂₈ is chosen from saturated or unsaturated linear C₇-C₂₂ alkyl radicals (for example n-octyl, n-decyl, n-hexadecyl, n-dodecyl or oleyl), branched alkyl radicals (for example isostearic) or cyclic alkyl radicals (for example cyclododecane or adamantane); CᵣC₁₈ alkylperfluoro radicals (for example the group of formula (CH₂)₂(CF₂)₉-CF₃); the cholesteryl radical or a cholesterol ester, for instance cholesteryl hexanoate; aromatic polycyclic groups, for instance naphthalene or pyrene.

Among these radicals, linear and branched alkyl radicals are more particularly preferred.

According to one embodiment of the invention, the hydrophobic radical R₂₈ may also comprise at least one alkylene oxide unit and in particular a polyoxyalkylene chain.

A polyoxyalkylene chain may be formed from ethylene oxide units and/or propylene oxide units and even more particularly be formed solely from ethylene oxide units.

The number of moles of oxyalkylene units may generally range from 1 to 30 mol, more particularly from 2 to 25 mol and even more particularly from 3 to 20 mol.

Among the AMPS® amphiphilic polymers that are suitable for use in the invention, mention may be made of:
crosslinked or noncrosslinked, neutralized or non-neutralized copolymers comprising from 15% to 60% by weight of AMPS units and from 40% to 85% by weight of (C₈-C₁₅)alkyl(meth)acrylamide units or of (C₈-C₁₆)alkyl (meth)acrylate units relative to the polymer, such as those described in patent application EP-A-0 750 899;
terpolymers comprising from 10 mol% to 90 mol% of acrylamide units, from 0.1 mol% to 10 mol% of AMPS units and from 5 mol% to 80 mol% of n(C₆-C₁₈)alkylacrylamide units relative to the polymer, such as those described in Pat. US-A-5 089 578.

As amphiphilic polymers that are suitable for use in the invention, mention may be made of polyoxyethylenated (crosslinked or noncrosslinked) copolymers of AMPS® and of alkyl methacrylates, and mixtures thereof.

As amphiphilic polymers that are suitable for use in the invention, mention may also be made of copolymers of totally neutralized AMPS® and of n-dodecyl, n-hexadecyl and/or n-octadecyl methacrylate, and also copolymers of AMPS and of n-dodecylmethacrylamide, which may be crosslinked or noncrosslinked.

Mention may also be made of crosslinked or noncrosslinked amphiphilic copolymers comprising, or even formed from:
2-acrylamido-2-methylpropanesulfonic acid (AMPS®) units of formula (lll) below:

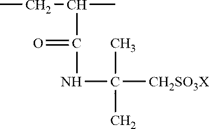

in which X may be a proton, an alkali metal cation, an alkaline-earth metal cation or an ammonium ion;
and units of formula (IV) below:

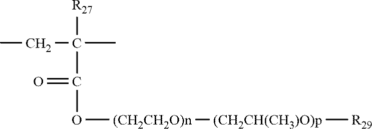

in which:
n and p, independently of each other, denote a number of moles and range from 0 to 30, in particular from 1 to 25 and more particularly from 3 to 20, with the proviso that n + p is less than or equal to 30, in particular less than 25 and more particularly less than 20;
R₂₇ has the same meaning indicated in the preceding formula (ll), and
R₂₉ denotes a linear or branched alkyl radical comprising from 7 to 22 and preferably from 12 to 20 carbon atoms.

In formula (lll), the cation X may more particularly denote sodium or ammonium. Mention may be made in particular of:
noncrosslinked polymers for which p = 0, n = 7 or 25, R₂₇ denotes methyl and R₂₉ represents a mixture of C12-C14 or C16-C18 alkyl, and
crosslinked polymers for which p = 0, n = 8 or 25, R₂₇ denotes methyl and R₂₉ represents a mixture of C16-C18 alkyl.

These polymers are described and synthesized in document EP 1 069 142.

These particular amphiphilic polymers may be obtained according to the standard free-radical polymerization processes in the presence of one or more initiators such as, for example, azobisisobutyronitrile (AIBN), azobisdimethylvaleronitrile, 2,2-azobis[2-amidinopropane] hydrochloride (ABAH), organic peroxides such as dilauryl peroxide, benzoyl peroxide, tert-butyl hydroperoxide, etc., mineral peroxide compounds such as potassium persulfate or ammonium persulfate, or H₂O₂ optionally in the presence of reducing agents.

These amphiphilic polymers may be obtained by free-radical polymerization in tert-butanol medium, in which they precipitate.

By using precipitation polymerization in tert-butanol, it is possible to obtain a size distribution of the polymer particles that is particularly favorable for its uses.

The reaction may be performed at a temperature of between 0 and 150° C. and preferably between 10 and 100° C., either at atmospheric pressure or under reduced pressure.

The reaction may also be performed under inert atmosphere, and preferably under nitrogen.

A polymer in accordance with the invention may be partially or totally neutralized with a mineral or organic base such as those mentioned above.

The molar percentage concentration of the units of formula (lll) and of the units of formula (IV) in an amphiphilic polymer according to the invention may vary as a function of the desired cosmetic application, the nature of the emulsion (oil-in-water or water-in-oil emulsion) and the rheological properties of the desired formulation.

It can range between 0.1 and 99.9 mol%.

The molar proportion of units of formula (IV) in an amphiphilic polymer according to the invention may preferably range from 0.1% to 50%, more particularly from 1% to 25%) and even more particularly from 3% to 10%.

The molar proportion of units of formula (IV) in an amphiphilic polymer according to the invention may preferably range from 50.1% to 99.9%, more particularly from 60% to 95% and even more particularly from 65% to 90%.

The distribution of the monomers in the polymers of the invention may be, for example, alternate, block (including multiblock) or random.

As a guide, and without this being limiting, mention may be made of the following commercial references: Aristoflex® HMS and Aristoflex® HMB sold by Clariant, these two references relating to crosslinked polymers.

Aristoflex® HMS is the name of the AMPS®/ethoxylated (25 EO) cetearyl methacrylate copolymer 80/20, crosslinked with trimethylolpropane triacrylate (TMPTA) or ammonium acryloyldimethyltaurate/stearate-25 methacrylate crosspolymer as the INCI name.

As the INCI name, Aristoflex® HMB is ammonium acryloyldimethyltaurate/Beheneth-25 methacrylate crosspolymer.

It is also possible to use, as hydrophobic AMPS copolymer, noncrosslinked AMPS® copolymers (Aristoflex® LNC or SNC), which are also effective in terms of stabilizing emulsions.

As the INCI name, Aristoflex® LNC is ammonium acryloyldimethyltaurate/Laureth-7 methacrylate copolymer.

As the INCI name, Aristoflex® SNC is ammonium acryloyldimethyltaurate/Steareth-8 methacrylate copolymer.

Among the acrylic polymers that may be combined with a combination in accordance with the invention, mention may also be made of neutralized crosslinked acrylic homopolymers or copolymers.

A preferred hydrophilic gelling agent of the present invention is ammonium polyacryloyldimethyl taurate, also known under the tradename, Hostacerin AMPS®, and commercially available from the supplier Clariant. It is also described as polyacrylamidomethylpropane sulfonic acid (AMPS®) partially neutralized with ammonia and highly cross-linked.

Other preferred hydrophilic gelling agents of the present invention are copolymers of AMPS and polyoxyethylene alkyl methacrylate (optionally cross-linked), and mixtures thereof such as ammonium acryloyldimethyltaurate/steareth-25 methacrylate crosspolymer, available under the tradenames Aristoflex HMS; ammonium acryloyldimethyltaurate/steareth-8 methacrylate crosspolymer, available under the tradenames Aristoflex SNC; and ammonium acryloyldimethyltaurate/VP copolymer, available under the tradenames Aristoflex AVC, Aristoflex JQD, Hostacerin SAF, all commercially available from the supplier Clariant.

According to a preferred embodiment, the cosmetic composition comprises at least one crosslinked and neutralized 2-acrylamido-2-methylpropanesulfonic acid homopolymer and at least one optionally crosslinked copolymer of AMPS and polyoxyethylene alkyl methacrylate.

According to a more preferred embodiment, the cosmetic composition comprises ammonium polyacryloyldimethyl taurate and ammonium acryloyldimethyltaurate/steareth-25 methacrylate crosspolymer.

Advantageously, the at least one acrylic polymer may be present in the cosmetic composition in an amount ranging from 0.1% to 5% by weight, such as from 0.15% to 3% by weight, or from 0.2% to about 2% by weight, relative to the total weight of the cosmetic composition according to the present invention.

### Cosmetic Active Ingredients

As a cosmetic composition, the composition of the present invention comprises at least one cosmetic active ingredient.

As examples of cosmetic active ingredient, mention can be made of vitamins (for example, ascorbyl glucoside, tocopheryl acetate), saccharomyces/xylinum/black tea ferment, vaccinium myrtillus fruit extract, saccharum officinarum (sugarcane) extract, citrus aurantium dulcis (orange) fruit extract, citrus limon (lemon) fruit extract, acer saccharum (sugar maple) extract, glycolic acid, hydroxypropyl tetrahydropyrantriol, caffin, secale cereale (rye) seed extract, sodium hyaluronate, betaine, dipeptide diaminobutyroyl benzylamide diacetate, etc.

According to one embodiment, the cosmetic active ingredient is present in an amount ranging from 0.1% to 15%, preferably from 1 to 12% by weight, more preferably from 3% to 10% by weight, relative to the total weight of the composition.

### Aqueous Phase

As an oil-in-water emulsion, the cosmetic composition of the present invention comprises a continuous aqueous phase.

Said aqueous phase is preferably present in an amount ranging from 10% to 99% by weight, more preferably from 20% to 90% by weight, and even more preferably from 50% to 85% by weight of the total weight of the composition.

Water is preferably present in the composition of the present invention in an amount ranging from 1% to 80% by weight, preferably from 5% to 77% by weight, more preferably from 10% to 75% by weight, relative to the total weight of the composition.

The continuous aqueous phase may comprise water, at least one organic solvent miscible with water or mixtures thereof.

Preferably, the continuous hydrophilic phase comprises at least one organic solvent miscible with water (at room temperature 25° C.) such as for example monoalcohols having from 2 to 6 carbon atoms such as ethanol, isopropanol; polyols having from 2 to 20 carbon atoms, preferably from 2 to 10 carbon atoms, and preferentially having from 2 to 6 carbon atoms, such as glycerin, propylene glycol, butylene glycol, pentylene glycol, hexylene glycol, caprylyl glycol, dipropylene glycol, diethylene glycol; glycol ethers (notably having from 3 to 16 carbon atoms) such as mono-, di- or tri- propylene glycol (C₁-C₄)alkyl ethers, mono-, di- or tri-ethylene glycol (C₁-C₄) alkyl ethers and mixtures thereof.

Preferably, the continuous hydrophilic phase of the composition of the present invention may comprise water and a polyol, preferably glycerin, propylene glycol, pentylene glycol.

### Fatty Phase

As an oil-in-water emulsion, the cosmetic composition of the present invention further comprises a dispersed fatty phase.

The fatty phase is present in an amount ranging from 1% to 50% by weight, preferably from 5% to 40% by weight, more preferably from 10% to 30% by weight, relative to the total weight of the composition of the present invention.

Said fatty phase preferably comprises at least one oil. The oil can be volatile or non-volatile.

The term “oil” means a water-immiscible non-aqueous compound that is liquid at room temperature (25° C.) and at atmospheric pressure (760 mmHg).

The term “non-volatile oil” means an oil that may remain on keratin materials at room temperature and atmospheric pressure for at least several hours and that especially has a vapour pressure of less than 10⁻³ mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Preferably, said oil is selected from hydrocarbonated, silicone or fluorinated oils.

The term “hydrocarbon-based oil” or “hydrocarbonated oil” means an oil formed essentially from, or even constituted by, carbon and hydrogen atoms, and optionally O and N atoms, and free of Si and F heteroatoms. Such oil can contain alcohol, ester, ether, carboxylic acid, amine and/or amide groups.

The term “silicone oil” means an oil containing at least one silicon atom, especially containing Si—O groups.

The term “fluorinated oil” means an oil containing at least one fluorine atom, Preferably, the oil is selected from hydrocarbonated oils, preferably non-volatile.

The oil can be, for example, present in an amount ranging from 0.01% to 50% by weight, preferably from 0.05% to 30% by weight, more preferably from 0.1% to 10% by weight, relative to the total weight of the composition.

### Structuring Agent(s)

Advantageously, the cosmetic composition of the present invention comprises at least one structuring agents with a melting point of less than or equal to 45° C.

Preferably, the structuring agents are selected from waxes, pasty compounds, and mixtures thereof.

The wax under consideration in the context of the present invention is generally a lipophilic compound that is solid at room temperature (25° C.), with a solid/liquid reversible change of state, having a melting point of greater than or equal to 30° C., preferably greater than or equal to 40° C., which may be up to 200° C. and in particular up to 120° C.

The term “pasty compounds” within the meaning of the present invention is understood to mean a lipophilic fatty compound with a reversible solid/liquid change in state which exhibits, in the solid state, an anisotropic crystalline arrangement and which comprises, at a temperature of 23° C., a liquid fraction and a solid fraction. In other words, the starting melting point of the pasty compound can be less than 23° C. The liquid fraction of the pasty compound, measured at 23° C., can represent from 9 to 97% by weight of the pasty compound. At 23° C., this liquid fraction preferably represents between 15 and 85% by weight, more preferably between 40 and 85% by weight of the pasty compound.

Within the meaning of the present invention, the melting point corresponds to the temperature of the most endothermic peak observed by thermal analysis (DSC) as described in Standard ISO 11357-3: 1999. The melting point of a pasty compound can be measured using a differential scanning calorimeter (DSC), for example the calorimeter sold under the name “MDSC 2920” by TA Instruments.

The measurement protocol is as follows:

A 5 mg sample of a pasty compound placed in a crucible is subjected to a first rise in temperature ranging from -20° C. to 100° C. at a heating rate of 10° C./minute, is then cooled from 100° C. to -20° C. at a cooling rate of 10° C./minute and, finally, is subjected to a second rise in temperature ranging from -20° C. to 100° C. at a heating rate of 5° C./minute. During the second rise in temperature, the variation in the difference in power absorbed by the empty crucible and by the crucible comprising the sample of pasty compound is measured as a function of the temperature. The melting point of the pasty compound is the value of the temperature corresponding to the tip of the peak of the curve representing the variation in the difference in power absorbed as a function of the temperature.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the latter to change from the solid state to the liquid state. The pasty compound is “in the solid state” when the whole of its mass is in the solid crystalline form. The pasty compound is “in the liquid state” when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by TA Instruments, with a rise in temperature of 5 or 10° C. per minute, according to Standard ISO 11357-3:1999.

The enthalpy of fusion of the pasty compound is the amount of energy necessary to change the pasty compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 30 to 100% by weight of the pasty compound, preferably from 50 to 100% by weight of the pasty compound, more preferably from 60 to 100% by weight of the pasty compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

Preferably, the melting point of the structuring agent is between 38° C. to 45° C.

Preferably, it is a wax having a melting point of between 38° C. to 45° C., such as linear esters.

Linear esters that are suitable for use in the present invention are preferably selected from the group consisting of stearyl stearate, tetradecyl tetradecanoate (INCI name: myristyl myristate), cetyl myristate, stearyl myristate, myristyl palmitate, stearyl palmitate, myristyl stearate, cetyl stearate, stearyl stearate and cetyl palmitate, and mixtures thereof.

Preferably, if presents, the structuring agent is present in an amount ranging from 0.1% to 5%, preferably from 0.2 to 2% by weight, more preferably from 0.5% to 1.5% by weight, relative to the total weight of the composition.

### Additional Adjuvants or Additives

The cosmetic composition of the present invention may comprise may also contain conventional cosmetic adjuvants or additives, for instance fragrances, chelating agents (for example, disodium EDTA), preserving agents (for example, chlorphenesin and phenoxy ethanol) and bactericides, additional surfactants, additional thickeners, fillers (for example, stearalkonium hectorite), pH regulators (for example citric acid or sodium hydroxide), and mixtures thereof.

According to a particularly preferred embodiment, the present invention provides a cosmetic composition in the form of an oil-in-water emulsion, comprising, relative to the total weight of the composition:
(i) from 0.5% to 3% by weight of at least one solid fatty alcohol having from 14 to 22 carbon atoms;
(ii) from 0.3% to 2% by weight of at least one amino acid surfactant selected from salts of glutamic acid; and
(iii) from 0.2% to 2% by weight of at least one gelling agent selected from crosslinked and neutralized 2-acrylamido-2-methylpropanesulfonic acid homopolymer and optionally cross-linked copolymers of AMPS and polyoxyethylene alkyl methacrylates, and mixtures thereof.

### Method and Use

According to the second aspect, the present invention relates to a non-therapeutic method for treating a keratin material, comprising the step of applying the composition according to the first aspect of the present invention to the keratin material.

In particular, the keratin material can be the skin.

Thus, in an embodiment of the second aspect, the present invention provides a non-therapeutic method for caring for the skin, comprising applying the cosmetic composition according to the first aspect of the present invention to the skin.

The following examples serve to illustrate the present invention without, however, being limiting in nature.

### EXAMPLES

### Example 1: Preparation of Cosmetic Compositions

Cosmetic compositions according to comparative formulas (Comp.) and inventive formula (Inv.) were prepared according to the amounts given in the table below. The amounts are given in % by weight of the total composition.

| INCI US | Inv.1 | Comp.1 | Comp.2 |
|---|---|---|---|
| | Wt.% | Wt.% | Wt.% |
| BUTYROSPERMUM PARKII (SHEA) BUTTER (FAIR FOR LIFE REFINED SHEA BUTTER from OLVEA) | 2 | 2 | 2 |
| DIISOPROPYL SEBACATE (DUB DIS from STEARINERIE DUBOIS) | 3 | 3 | 3 |
| CHLORPHENESIN (MACROCIDE-OL from MACROCARE) | 0.2 | 0.2 | 0.2 |
| SODIUM STEAROYL GLUTAMATE (EUMULGIN® SG from BASF) | 0.35 | 0.35 | 0.35 |
| PENTYLENE GLYCOL (PENTIOL from MINASOLVE) | 2 | 2 | 2 |
| WATER | QS 100 | QS 100 | QS 100 |
| GLYCERIN (GLYCERINE CP from VVF) | 7 | 7 | 7 |
| PHENOXYETHANOL (GLYSOLV EPHL from HUNTSMAN) | 0.39 | 0.39 | 0.39 |
| CAPRYLYL GLYCOL (CLEANBIO® -CG from KOLON LIFE SCIENCE) | 0.3 | 0.3 | 0.3 |
| DIMETHICONE (and) DIMETHICONE/VINYL DIMETHICONE CROSSPOLYMER (KSG 16 from SHIN ETSU) | 2 | 2 | 2 |
| SODIUM HYALURONATE (SODIUM HYALURONATE (HA-T) from BLOOMAGE FREDA BIOPHARM) | 0.03 | 0.03 | 0.03 |
| CAFFEINE (CAFEINE ANHYDRE NATURELLE POUDRE (EX COFFEE) from TESTA) | 0.2 | 0.2 | 0.2 |
| BETAINE (AMINOCOAT from ASAHI KASEI) | 2 | 2 | 2 |
| DISODIUM COCOYL GLUTAMATE (PLANTAPON® ACG LC from BASF) | 1 | 1 | 1 |
| SACCHAROMYCES/XYLINUM/BLACK TEA FERMENT (and) GLYCERIN (KOMBUCHKA™ from SEDERMA (CRODA)) | 3.5 | 3.5 | 3.5 |
| SECALE CEREALE (RYE) SEED EXTRACT (COHELISS® GR from SILAB) | 0.1 | 0.1 | 0.1 |
| POLYGLYCERYL-6 DISTEARATE (and) JOJOBA ESTERS (and) CETYL ALCOHOL (and) POLYGLYCERYL-3 BEESWAX (EMULIUM® MELLIFERA from GATTEFOSSE) | 3 | 3 | 3 |
| CETYL ALCOHOL (LANETTE® 16 from BASF) | 0.4 | - | 0.4 |
| PENTAERYTHRITYLTETRAETHYLHEXANOATE (DUB PTO | 1 | 1 | 1 |
| from STEARINERIE DUBOIS) | | | |
| BEHENYL ALCOHOL (LANETTE®22 from BASF) | 0.9 | - | 0.9 |
| XANTHAN GUM (KELTROL® CG from CP KELCO) | 0.3 | 0.3 | 0.3 |
| SODIUM HYALURONATE (LOW MOLECULAR WEIGHT SODIUM HYALURONATE (HA-TLM) from BLOOMAGE FREDA BIOPHARM) | 0.5 | 0.5 | 0.5 |
| TOCOPHERYL ACETATE (DL ALPHA TOCOPHERYL ACETATE from DSM NUTRITIONAL PRODUCTS) | 0.05 | 0.05 | 0.05 |
| DIMETHICONE (and) DIMETHICONOL (BRB 1740 from BRB INTERNATIONAL) | 2 | 2 | 2 |
| DISODIUM EDTA (DISSOLVINENA-2 from AKZO NOBEL) | 0.1 | 0.1 | 0.1 |
| DI-C12-13 ALKYL MALATE (COSMACOL EMI from SASOL) | 2 | 2 | 2 |
| STEARALKONIUM HECTORITE (BENTONE® 27 V CG from ELEMENTIS) | 0.5 | 0.5 | 0.5 |
| ASCORBYL GLUCOSIDE (V2G from MACROCARE) | 0.2 | 0.2 | 0.2 |
| MYRISTYL MYRISTATE (TEGOSOFT® MM from EVONIK GOLDSCHMIDT) | 1 | 1 | 1 |
| AMMONIUM ACRYLOYLDIMETHYLTAURATE/STEARETH-25 METHACRYLATE CROSSPOLYMER (ARISTOFLEX® HMS from CLARIANT) | 0.49 | 0.49 | - |
| SODIUM HYDROXIDE (32% SODIUM HYDROXIDE from JIANGSU KOLOD FOOD INGREDIENTS) | 0.01 | 0.01 | 0.01 |
| AMMONIUM POLYACRYLOYLDIMETHYLTAURATE (HOSTACERIN AMPS from CLARIANT) | 0.21 | 0.21 | - |
| POLYACRYLATE CROSSPOLYMER-6 (SEPIMAX™ ZEN from SEPPIC) | - | - | 0.7 |
| HYDROXYPROPYL TETRAHYDROPYRANTRIOL (MEXORYL SCN from CHIMEX) | 3 | 3 | 3 |
| DIPEPTIDE DIAMINOBUTYROYL BENZYLAMIDE DIACETATE (SYN-AKE from DSM) | 0.01 | 0.01 | 0.01 |
| PROPANEDIOL (ZEMEA® PROPANEDIOL from DUPONT TATE AND LYLE BIO PRODUCTS) | 3 | 3 | 3 |

Composition of comparative example 1 does not comprise solid fatty alcohol.

Composition of comparative example 2 does not comprise acrylic polymer as hydrophilic gelling agent.

### Preparation Process

The compositions listed above were prepared as follows, taking the composition of invention formula 1 as an example:
1. Homogenizing the aqueous phase and the oil phase at about 75° C. and 80° C., respectively.
2. Introducing the oil phase into aqueous phase, keep stirring at high temperature to complete the emulsifying.
3. Adding the polymers (AMPS, HMS, HA, Xanthan gum) sequentially, keep stirring to make them dispersed evenly, and
4. Lowering the temperature to add the silicones and actives.

### Example 2: Evaluation of Cosmetic Compositions

The stability, texture and skin feeling of each cosmetic composition prepared in Example 1 were measured.

### Stability

The stability tests of the cosmetic compositions according to invention and comparative formulas at 40° C., 45° C., and 65° C. were conducted using Binder oven (USA), by leaving the invention and comparative formulas in the oven for 3 months.

The stability tests at 4° C. stability for two months were conducted using Zhongke Meiling refrigerator (YC-260L, China), by leaving the cosmetic compositions according to invention and comparative formulas in the refrigerator for 3 months.

The light stability tests for 24 hours were conducted using ATLAC (AMETEK Measurement and Calibration Technologies).

Lastly, the freezing-thaw stability tests were conducted for 10 cycles using Binder over (USA). In each cycle, the temperature will be changed gradually from 20° C. to -20° C. in 24 hours.

### Texture

The texture was evaluated according to the viscosity of each of the cosmetic compositions of invention formulas and compositions of comparative formulas.

The viscosity measurement is generally performed at 25° C., using a Rheomat RM180 viscometer equipped with a M3 spindle, the measurement being performed after 10 minutes of rotation of the spindle in the composition (after which time stabilization of the viscosity and of the spin speed of the spindle are observed), at a shear rate of 200 rpm.

### Skin Feeling

The cosmetic compositions of inventive and comparative formulas were given to 5 consumers to be applied on the skin, and score of easy spreading, as well as fresh and moisturizing feeling were given by the consumers:
1: very poor;
2: little effect, not acceptable;
3: acceptable;
4: good effect;
5: very good effect, very easy to pick up and spread, fresh on the skin.

The result of the stability, viscosity, usage and cosmetic properties of the compositions according to comparative formula and inventive formulas were listed below.

| Test | | Inv. 1 | Comp.1 | Comp. 2 |
|---|---|---|---|---|
| Stability test at different temperatures in 3 months | 4° C. | pass | not pass | not pass |
| | 40° C. | pass | not pass | not pass |
| | 45° C. | pass | not pass | not pass |
| | 65° C. | pass | not pass | not pass |
| Light stability test | | pass | not pass | not pass |
| Freezing-shaw stability test | | pass | not pass | not pass |
| Viscosity (UD (M3)) | | 33 | Too low | Too low |
| Easy spreading | | 5 | 2 | 2 |
| Fresh and moisturizing feeling | | 5 | 2 | 2 |

The cosmetic composition of inventive formula 1 can be easily spread on the skin, and provides to the skin fresh and moisturizing feeling. In addition, the cosmetic composition of inventive formula 1 is stable and presents a good texture.

1. A cosmetic composition , comprising:
(i) at least one solid fatty alcohol;
(ii) at least one amino acid surfactant; and
(iii) at least one gelling agent selected from hydrophilic acrylic homopolymers and amphiphilic acrylic polymers,
wherein the cosmetic composition is in the form of oil-in-water emulsion.

2. The cosmetic composition of claim 1, wherein the solid fatty alcohol is of structure R—OH with R denoting a saturated or unsaturated, linear alkyl group, optionally substituted with one or more hydroxyl groups, comprising from 14 to 30 carbon atoms.

3. The cosmetic composition of claim 1 , wherein the solid fatty alcohol is present in an amount ranging from 0.1% to 10% by weight relative to the total weight of the composition.

4. The cosmetic composition of claim 1 , wherein the amino acid surfactant is of formula (A), 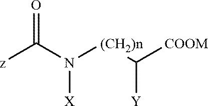
wherein in formula (A):
Z represents a saturated or unsaturated, linear or branched hydrocarbon group having 8 to 22 carbon atoms,
X is hydrogen or methyl group,
n is 0 or 1,
Y is selected from hydrogen, —CH₃, —CH(CH₃)₂, —CH₂CH(CH₃)₂, —CH(CH₃)CH₂CH₃, —CH₂C₆H₅, —CH₂C₂H₄OH, —CH₂OH, —CH(OH)CH₃, —(CH₂)₄NH₂, —(CH₂)₃NHC(NH)NH₂, —CH₂C(O)O^{—}M⁺, —(CH₂)₂C(O)OH, —(CH₂)₂C(O)O^{—}M⁺, and
M is a salt-forming cation.

5. The cosmetic composition of claim 4, wherein the amino acid surfactant is selected from the group consisting of salts of alanine, arginine, aspartic acid, glutamic acid, glycine, isoleucine, leucine, lysine, phenylalanine, serine, tyrosine, valine, sarcosine, and any mixture thereof.

6. The cosmetic composition of claim 1 , wherein the amino acid surfactant is present in an amount ranging from 0.1% to 5% by weight relative to the total weight of the composition.

7. The cosmetic Cosmetic composition of claim 1 as , wherein the hydrophilic gelling agent is selected from AMPS homopolymers and amphiphilic acrylic polymers.

8. The cosmetic composition of claim 1 , wherein the hydrophilic gelling agent is selected from i) AMPS homopolymers comprising, randomly distributed:
a) from 90% to 99.9% by weight of units of general formula (I) below: 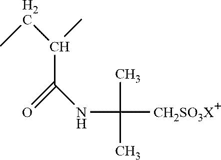
in which X⁺ denotes a proton, an alkali metal cation, an alkaline-earth metal cation or the ammonium ion, with the proviso that not more than 10 mol% of the cations X⁺ are protons H⁺;
b) from 0.01% to 10% by weight of crosslinking units derived from at least one monomer containing at least two olefinic double bonds; the weight proportions being defined relative to the total weight of the polymer; and
ii) optionally cross-linked copolymers of AMPS and polyoxyethylene alkyl methacrylate.

9. The cosmetic composition of claim 1 , wherein the cosmetic composition comprises at least one crosslinked and neutralized 2-acrylamido-2-methylpropanesulfonic acid homopolymer and at least one optionally crosslinked copolymer of AMPS and polyoxyethylene alkyl methacrylate .

10. The cosmetic composition of claim 1 , wherein the acrylic polymer is present in an amount ranging from 0.1% to 5% by weight relative to the total weight of the composition.

11. The cosmetic composition of claim 1 , wherein the composition comprises an aqueous phase in an amount ranging from 10% to 99% by weight relative to the total weight of the composition.

12. The cosmetic composition of claim 1 , wherein the composition comprises a fatty phase in an amount ranging from 1% to 50% by weight relative to the total weight of the composition.

13. A cosmetic composition , comprising, relative to the total weight of the composition:
(i) from 0.5% to 3% by weight of at least one solid fatty alcohol having from 14 to 22 carbon atoms;
(ii) from 0.3% to 2% by weight of at least one amino acid surfactant selected from salts of glutamic acid; and
(iii) from 0.2% to 2% by weight of at least one hydrophilic gelling agent selected from crosslinked and neutralized 2-acrylamido-2-methylpropanesulfonic acid homopolymer and optionally cross-linked copolymers of AMPS and polyoxyethylene alkyl methacrylates, and mixtures thereof, wherein
the composition is in the form of an oil-in-water emulsion.

14. A non-therapeutic method for caring for the skin, comprising applying the cosmetic composition of claim 1 to the skin.

15. The cosmetic composition of claim 4, wherein
Z represents a linear or branched C₈ to C₂₂ alkyl or alkenyl group,
X is a hydrogen or methyl group,
n is 0,
Y is selected from hydrogen, —(CH₂)₂C(O)OH, —(CH₂)₂C(O)O^{—}M⁺, and
M is a salt-forming cation, selected from the group consisting of sodium, potassium, ammonium and triethanolammonium.

16. The cosmetic composition of claim 9, comprising ammonium polyacryloyldimethyl taurate and ammonium acryloyldimethyltaurate/steareth-25 methacrylate crosspolymer.
